(19) United States
(12) Patent Application Publication
(10) Pub. No.: US 2020/0171256 A1
(43) Pub. Date: Jun. 4, 2020

(54) Intubation Systems and Methods

(71) Applicant: AI Medical Devices, Inc., Williamston, MI (US)

(72) Inventors: Richard B. Schwartz, Evans, GA (US); John D. Schwartz, Williamston, MI (US)

(21) Appl. No.: 16/698,839

(22) Filed: Nov. 27, 2019

Related U.S. Application Data
(60) Provisional application No. 62/773,615, filed on Nov. 30, 2018.

(51) Int. Cl. A61M 16/04

(52) U.S. Cl. CPC A61M 16/0463; A61M 16/201; A61M 16/0497

(57) ABSTRACT

An intubation device for insertion into a patient's trachea is provided. The intubation device includes a catheter and an endotracheal tube coaxially positioned over the catheter, where an interior surface of the endotracheal tube and an exterior surface of the catheter define an interstitial space. The device also includes a hub that has a gas supply port configured to connect the body of the hub with a gas supply source, a first port in a proximal end and a second port in a distal end. Each of the first port and the second port is configured to receive the catheter and the hub also includes a connector configured to connect to the endotracheal tube such that the interstitial space is in fluid communication with the body and the gas supply port.

### CROSS REFERENCE

The present application relies on U.S. Patent Provisional Application No. 62/773,615, entitled “Intubation Systems and Methods” and filed on Nov. 30, 2018, for priority, which is herein incorporated by reference in its entirety.

### FIELD

The present invention relates generally to medical intubation systems and, more specifically, to an intubation device and guide system for intubating a patient by accessing the patient's trachea.

### BACKGROUND

Tracheal intubation, usually simply referred to as intubation, is the placement of a flexible plastic tube into the trachea, commonly referred to as the windpipe, to maintain an open airway or to serve as a conduit through which to administer certain drugs. It can be performed in critically injured, ill, or anesthetized patients to facilitate ventilation of the lungs, including mechanical ventilation, and to prevent the possibility of asphyxiation or airway obstruction.

The most widely used route is orotracheal, in which an endotracheal tube is passed through the mouth and vocal apparatus into the trachea. In a nasotracheal procedure, an endotracheal tube is passed through the nose and vocal apparatus into the trachea.

Because it is an invasive and uncomfortable medical procedure, intubation can be performed after administration of general anesthesia and a neuromuscular-blocking drug. It can, however, be performed in a patient who is awake with by using a local or topical anesthesia or in an emergency without any anesthesia at all. Intubation is normally facilitated by using a conventional laryngoscope or a flexible fiber-optic bronchoscope to identify the vocal cords and pass the tube between them into the trachea instead of into the esophagus.

U.S. Pat. No. 4,069,820 to Berman teaches an intubating pharyngeal airway having a side access for passage of a tube on the said airway comprising a flanged stop at the proximal end, a curved airway central tubular member and a distal ball tip adapted to fit into the vallecular. The side opening may be expanded or closed by means of either a hinge on the opposite side wall of the tube or by a cap or insert closure.

U.S. Pat. No. 4,612,927 issued to Kruger relates to an instrument for keeping clear the upper respiratory passages and for performing intubations, in which a tube which is to be inserted may have its distal extremity moved as far as into the windpipe via a passage acting as a guide. The passage is constructed as a channel extending within the instrument shaft, whereas the distal instrument extremity comprises a head before which terminates the channel and which is intended to be placed in contact against the larynx upon inserting the instrument. The instrument head acting as a stop will thus limit the maximum depth of insertion.

U.S. Pat. No. 5,203,320 issued to Augustine discloses a tracheal intubation guide having a tubular member with a curved forward end shaped to follow the curvature of the back of the tongue and throat of a patient, and a rear end for projecting out through the mouth of the patient, and an anterior guide surface extending along at least part of the length of the member to its forward end for guiding the member into the throat into a position opposite the opening into the larynx. The tubular member has a through bore for holding an endotracheal tube, and the guide surface has a forward edge of concave shape for engaging the front of the epiglottis and seating over the hyoepiglottic ligament when the member is accurately positioned. Correct positioning can be detected by external palpation of the neck.

U.S. Pat. No. 5,053,166 issued to Gomez discloses an intubating assembly used to position an intubation tube having a distal end, a proximal end and a generally resilient tubular configuration, into a trachea of a patient. The intubating assembly is described as having a guide assembly that receives the intubation tube therein and conforms the intubation tube to its configuration. The guide assembly includes first and second introduction segments hingedly coupled to one another and positionable between a closed orientation, which defines a generally curved configuration of the guide assembly, and an open orientation, which defines a generally straight configuration of the guide assembly. The intubating assembly further includes a positioning assembly structured to selectively position the first and second introduction segments between the open orientation. The intubation tube is generally straightened to facilitate direct introduction thereof into an airway of the patient to a point posterior of a tip of an epiglottis of the patient and the closed orientation. The intubation tube is generally curved in order to angle the distal end thereof towards the trachea of the patient and thereby introduce the intubation tube directly into the trachea of the patient.

U.S. Pat. No. 6,539,942 issued to Schwartz et al., hereby incorporated herein by reference in its entirety, describes an endotracheal intubation device having a series of interlinked, truncated ring-like elements disposed along the distal portion of the tube and a handgrip for controlling the degree of bend in the distal end of the device. An imaging device, such as a nasopharyngoscope, can be inserted through the intubation device to visualize the patient's vocal cords during the intubation procedure. The endotracheal intubation device uses a scissors mechanism without pulleys to bend the distal end of the device.

U.S. Pat. No. 8,820,319 issued to Schwartz et al., hereby incorporated herein by reference in its entirety, describes a guide adapted for facilitating insertion of a medical device into the trachea of a patient is disclosed. The guide includes an integral curved-shaped member having at least a first leg at one end of the guide. The curved-shaped member essentially includes an outside curved side defining a concave groove. A first angle is defined on the first leg configured and dimensioned to allow for at least the first leg to pass through the mouth and into the trachea of the patient. Insertion of the guide into the trachea allows for elevation of the tongue and surrounding soft tissue of the patient thereby forming an air space that allows for passage of a medical device.

Airway and respiratory complications are leading causes of morbidity in anesthesia, emergency medicine and critical care. Hypoxia and failure to intubate the trachea are also leading causes of malpractice claims. Video laryngoscopes have been developed with the hope of improving outcomes compared to direct laryngoscopes. Unfortunately, improvements in video laryngoscopes have not resulted in the desired improvements in success rates of intubation procedures. The use of a video laryngoscope in intubation procedures has some disadvantages, such as, secretions and blood blocking the laryngeal view of the camera of the laryngoscope, the time for the intubation procedure being limited to the amount of time before the oxygen level drops in the patient, and fogging of the optical window limiting the laryngeal view. Fogging also limits the ability to guide an endotracheal tube through the vocal cords in a patient.

Hence, there is a need for an endotracheal intubation device that can provide gas and suction at a distal end of an endotracheal intubation device that is to be inserted into a patient's trachea.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses an intubation device configured to be inserted into a patient's trachea, the intubation device comprising: a first tube; a second tube configured to be positioned over the first tube, wherein an interior surface of the second tube and an exterior surface of the first tube define an interstitial space; and a hub comprising a proximal end, a distal end and a body, wherein the hub comprises: a gas supply port configured to connect the body of the hub with a gas supply source; a first port in the proximal end and a second port in the distal end, wherein each of the first port and the second port is configured to receive the first tube and wherein the first tube passes through the body of the hub between the first port and the second port; a first connector configured to connect to the second tube such that the interstitial space is in fluid communication with the body and the gas supply port.

The first port may be configured to create a friction-based seal around the exterior surface of the first tube.

The second port may be configured to create a friction-based seal around the exterior surface of the first tube.

Optionally, the first tube is a catheter and the second tube is an endotracheal tube.

Optionally, the catheter comprises an angled tip configured to be insertable into the patient's trachea via the patient's mouth.

Optionally, the hub further comprises a control valve for controlling at least one of a gas flow rate or a level of gas pressure in the body. The control valve may be configured to limit a gas flow rate to less than 10 liters per minute. The control valve may be configured to release gas from the body when a pressure of the gas exceeds a predefined threshold value.

Optionally, the intubation device further comprises a suction control port attached to a proximal end of the first tube. Optionally, the suction control port comprises a first connector portion configured to be attached to suction source and an opening configured to direct suction from the suction source through the first tube when the opening is covered and configured to direct suction from the suction source to an external environment when the opening is uncovered. The suction control port may be configured to be covered by physically blocking said opening. Optionally, when the suction control port is closed, the suction control port is configured to direct suction through the first tube in order to remove fluids from an oropharynx of the patient.

Optionally, the gas supply source is configured to supply oxygen.

Optionally, the body of the hub is configured to fluidly separate an interior of the first tube from gas supplied by the gas supply source.

The present specification also discloses a method of operating an intubation device configured to be inserted into a patient's trachea, wherein the intubation device comprises a catheter, an endotracheal tube configured to be coaxially positioned over the catheter, wherein an interior surface of the endotracheal tube and an exterior surface of the catheter define an interstitial space, and a hub comprising a proximal end, a distal end and a body, wherein the hub comprises a gas supply port configured to connect the body of the hub with a gas supply source, a first port in the proximal end and a second port in the distal end, wherein each of the first port and the second port is configured to receive the catheter and wherein the catheter passes through the body of the hub between the first port and the second port, and a first connector configured to connect to the endotracheal tube such that the interstitial space is in fluid communication with the body and the gas supply port, the method comprising: inserting an insertion guide body into the patient's mouth; inserting the catheter sheathed by the endotracheal tube into the patient's trachea using the insertion guide body; supplying oxygen to a distal tip of the catheter by passing oxygen from the gas supply source, through the gas supply port, into the body of the hub, and through the interstice; maneuvering the catheter to guide the endotracheal tube past the larynx and the vocal cords of the patient; inflating a balloon at a distal tip of the endotracheal tube in order to fix a position of the endotracheal tube at a desired location; and withdrawing the catheter and the insertion guide body from the patient's body, leaving only the endotracheal tube fixed in the desired location remaining within the patient's body.

Optionally, the first port is configured to create a friction-based seal around the exterior surface of the catheter and wherein the second port is configured to create a friction-based seal around the exterior surface of the catheter.

Optionally, the distal tip of the catheter comprises an angled tip configured to be insertable into the patient's trachea via the patient's mouth.

Optionally, the hub further comprises a control valve for controlling at least one of a flow rate or a level of gas pressure in the body.

Optionally, the method further comprises using the control valve to release gas from the hub when the flow rate of gas exceeds 10 liters per minute.

Optionally, the intubation device further comprises a suction control port attached to a proximal end of the catheter, wherein the suction control port comprises a first connector portion configured to be attached to suction source and a separate opening. Optionally, the method further comprises modifying an amount of suction being applied from the suction source via the catheter by covering the opening, wherein the suction control port is configured to direct suction from the suction source through the catheter when the opening is covered and configured to direct suction from the suction source to an external environment when the opening is uncovered.

A distal end of the insertion guide body may comprise a light source and a camera.

Optionally, the balloon is inflated only after the endotracheal tube has extended past the patient's larynx and vocal cords.

Optionally, the method further comprises steering the distal tip of the catheter by gripping and moving the suction control port attached to the catheter.

These and other features, advantages and objects of the various embodiments will be better understood with reference to the following specification and claims.

### BRIEF DESCRIPTION OF THE DRAWINGS

These and other features and advantages of the present specification will be further appreciated, as they become better understood by reference to the following detailed description when considered in connection with the accompanying drawings:

FIG. 1 illustrates an intubation system, in accordance with an embodiment of the present specification;

FIG. 2A illustrates a catheter of the intubation system shown in FIG. 1, in accordance with an embodiment of the present specification;

FIG. 2B illustrates a close-up view of a connector and hub as shown in FIG. 2A;

FIG. 2C illustrates another close-up view of the connector and hub shown in FIG. 2B;

FIG. 2D illustrates yet another close-up view of the connector and hub shown in FIG. 2B;

FIG. 2E illustrates a transparent view of a hub with a steerable catheter, in accordance with an embodiment of the present specification;

FIG. 3A illustrates the intubation device, in accordance with an embodiment of the present specification;

FIG. 3B illustrates another view of the intubation device shown in FIG. 3A;

FIG. 3C illustrates another view of the intubation device shown in FIG. 3B;

FIG. 3D illustrates a catheter and an endotracheal tube of the intubation device shown in FIG. 3C separately;

FIG. 4A shows an endotracheal intubation guidance system that may be used with the with the intubation insertion device of the present specification;

FIG. 4B shows another view of the endotracheal intubation guidance system shown in FIG. 4A;

FIG. 5 shows a side view of an endotracheal intubation guidance system that may be used with the with the intubation insertion device of the present specification; and

FIG. 6 is a flowchart illustrating the method of operating the intubation device, in accordance with an embodiment of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

As used herein, the indefinite articles “a” and “an” mean “at least one” or “one or more” unless the context clearly dictates otherwise.

The intubation device described in the present specification is an easy to use device that is designed to improve intubation success rates and to reduce complications associated with endotracheal intubation. The intubation device comprises a catheter, which is designed to be used with a channeled video laryngoscope, in combination with a hub. The intubation device provides a precise suction catheter and eliminates secretions and blood as an operator advances the device within a patient's body as part of an intubation procedure. The intubation device of the present specification can provide a high flow of gas, such as, but not limited to oxygen, at the patient's larynx via the catheter and hub, and also comprises an integrated pressure relief valve in the hub that reduces the possibility of causing barotrauma to the patient's lungs.

In an embodiment, the gas flow is provided, via the hub, to an interstice between the catheter/suction tube and an endotracheal tube attached to the intubation device. In an embodiment, high flow oxygen can be supplied as the gas to increase the time before desaturation. The supply of the high flow gas at distal end of the catheter, e.g., within the larynx, can reduce the relative humidity of the oropharynx which can reduce or eliminate fogging of the optical window of the camera. The novel configuration of the suction catheter allows it to have the additional function as a guide tube or intubating bougie. The angled tip of the catheter allows for steering of the suction catheter through the vocal cords allowing for a guide for difficult intubation.

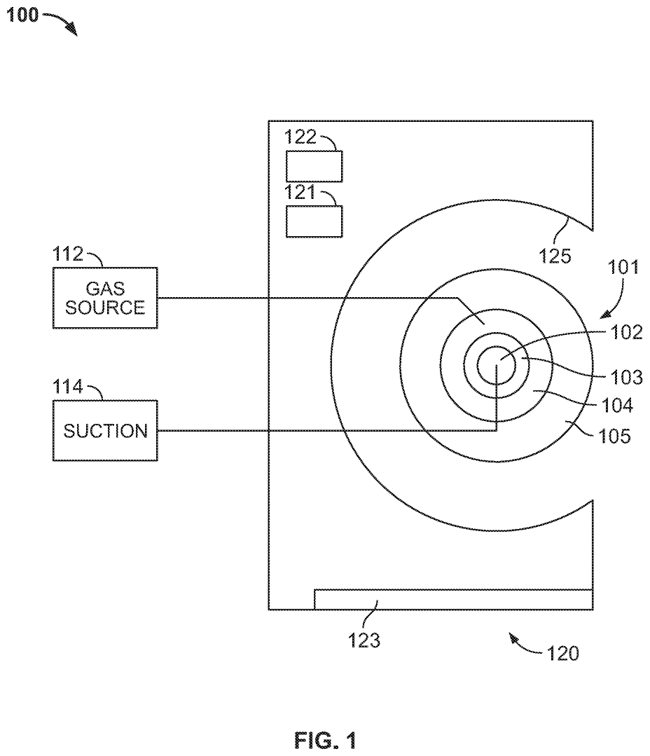

FIG. 1 illustrates an intubation system, in accordance with an embodiment of the present specification. FIG. 1 shows a schematic view of an intubation system 100 comprising an insertion body 120 and an insertion device or catheter 101 supported and guided by the insertion body 120. In an embodiment, the insertion device 101 comprises an inner suction flexible tube 103, which may be referred to herein as a catheter, and an outer flexible tube 105, which may be referred to herein as an endotracheal tube.

The inner, suction tube 103 comprises a hollow interior forming an enclosed fluid channel 102 that forms a suction port at a distal end that is inserted into a patient's body. Fluid, or a gas, can be vacated from inside the patient's body via the distal end of the suction tube 103, by using a suction source 114 fluidly connected to the channel 102. The suction source 114 provides suction to evacuate fluids accumulated in a patient's body via the distal end of the suction tube 103. A gas source 112 is coupled to the device 101 for providing a gas, such as, but not limited to, oxygen from a hospital supply to the patient. In an embodiment, the gas source provides oxygen at a flow rate of at least 10 L/min. The outer tube 105 is positioned outside (enveloping) the inner tube 103 to form an interstice 104 between the two tubes 103, 105. In various embodiments, the interstice 104 operates as a gas channel 104 to provide gas from the gas source 112 to a distal end of the device 101. In an embodiment, the gas channel 104 has a greater volume than the fluid channel 102 formed by the suction tube 103. In an embodiment, a distal port of the gas channel 104 completely encircles or surrounds a radially inward designed distal end of the suction tube 103 which forms the suction port for evacuating fluids/gas via the fluid channel 102.

In an embodiment, a diameter of the gas channel 104 may be obtained by subtracting the outer diameter of the tube 103 from an inner diameter of the tube 105, as can be seen in FIG. 1. Hence, in embodiments, the diameter of the gas channel 104 is greater than the diameter of the inner tube 103 by a factor of more than 1.5, 2.0, 5.0 or any increment therein. In an embodiment, a radius of the fluid channel 102 of the tube 103 is less than a radial dimension of the gas channel 104. The gas channel 104 being greater, in linear dimension and volume, than the suction fluid channel 102 allows the gas channel 104 to supply a greater volume of gas for assisting the patient's breathing or for inflating the patient's tissue to increase visibility, than a volume of fluid removed from a patient's body by the suction fluid channel 102. In various embodiments, the suction fluid channel 102 can provide suction simultaneously with the gas channel 104 supplying a gas to the patient.

In various embodiments, the insertion body 120 acts as a guide for inserting the catheter/insertion device 101 into a patient's body, usually via the patient's mouth. The insertion body 120 comprises a fixed blade 123 for guiding the catheter 101 into the patient's body. In an embodiment, the blade 123 is rigid and does not bend or significantly deflect under the forces required to insert the blade 123 into the soft tissue of the patient's mouth and trachea. The insertion body 120 has a proximal end closer to a medical professional operating the intubation system 100 and a distal end that is designed to be inserted into the patient. The blade 123 is located at the distal end of the insertion body 120. In embodiments, the insertion body 120 may be designed as a solid body or a hollow body. The insertion body 120 has a generally cylindrical shaped channel 125 in which the catheter 101 is received and guided to the oropharynx and trachea of the patient. The guide channel 125 is open at both the distal end, the proximal end of the insertion body 120, and comprises an opening all along one side resulting in a generally c-shaped cross-section. A light source 121 and camera 122 are located at the distal end of the insertion body 120. Both of the camera 122 and light source 121 are electrically connected to a controller (not shown in the FIG.) located at the proximal end of the body 120 or otherwise available to the medical professional. In an embodiment, the camera 122 is a solid-state imager, such as, but not limited to a charge-coupled device (CCD) camera. In an embodiment, the camera 122 sends a video signal through electrical connections in the guide body 120 to a viewing device (not shown in the FIG.) for displaying images obtained by the camera 122 of the patient's tissues at the distal end of the insertion body 120. In embodiments, the viewing device is a display that may be located at the proximal end of the insertion body 120, or may be located remote to the insertion body 120.

In embodiments, the light source 121 comprises light emitting diodes (LED) coupled within fiber optic cable, a light transmissive media and the like, for reaching the distal end of the insertion body 120 to illuminate the patient's tissues around insertion body 120 distal end in order for the camera 122 to capture images of said tissues. In various embodiments, the outer tube 105 functions as an endotracheal tube. The guide channel 125 is sized to fit the inner tube 103 and the outer/endotracheal tube 105 enveloping the inner/suction tube 103 which can slide within the channel 125 to exit the distal end of the channel 125 along an outside of the inner tube 103 and into a trachea of the patient.

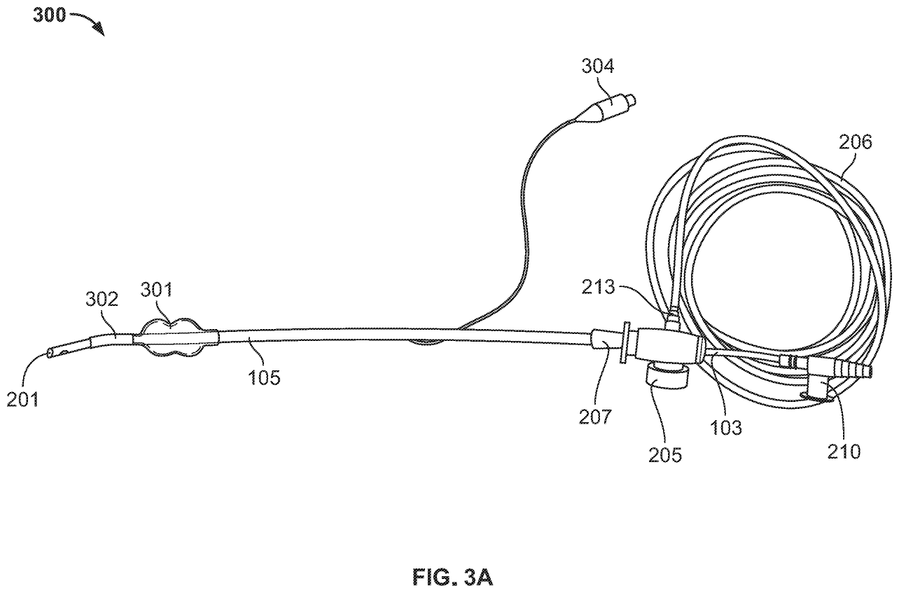

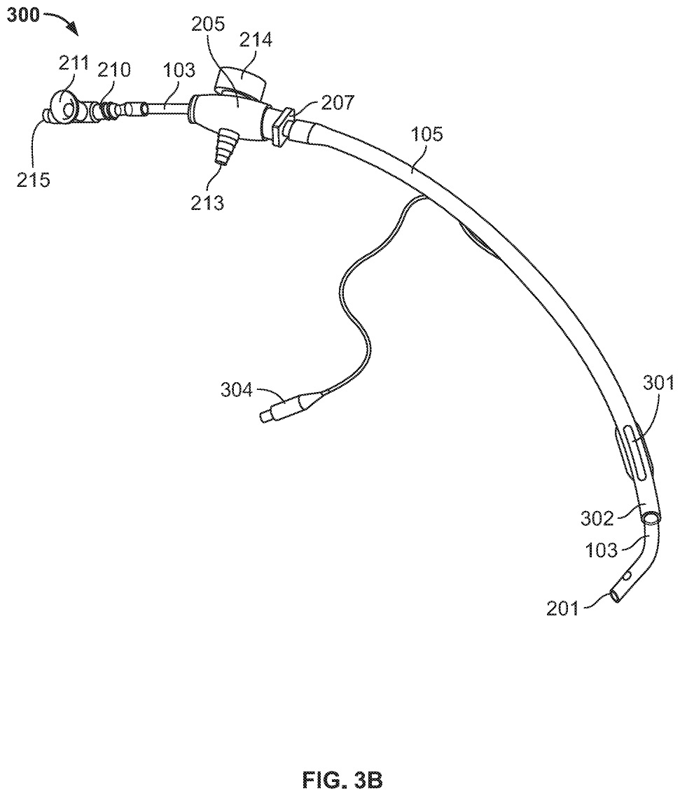

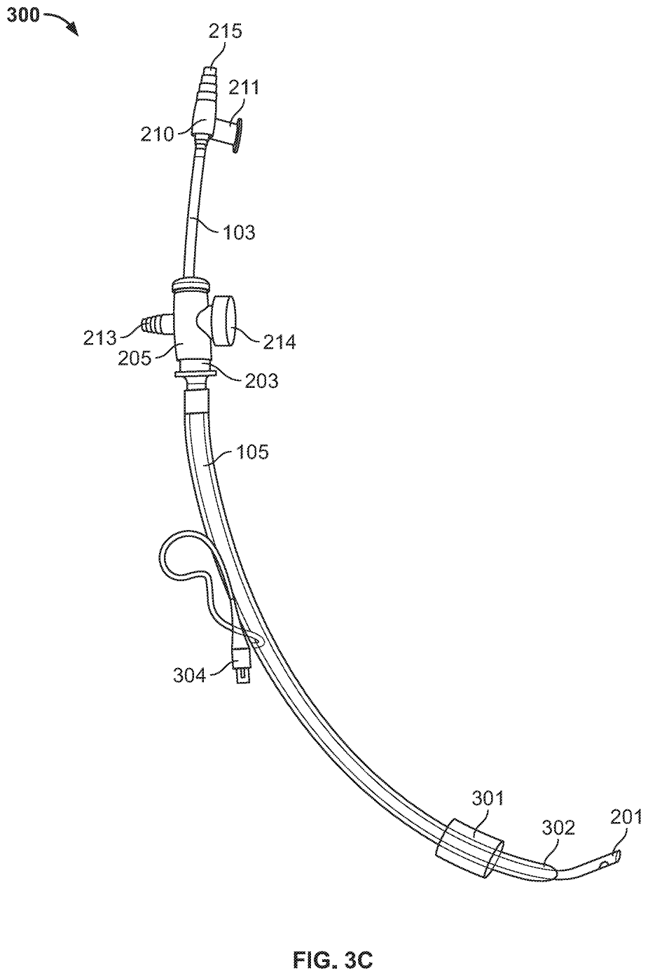

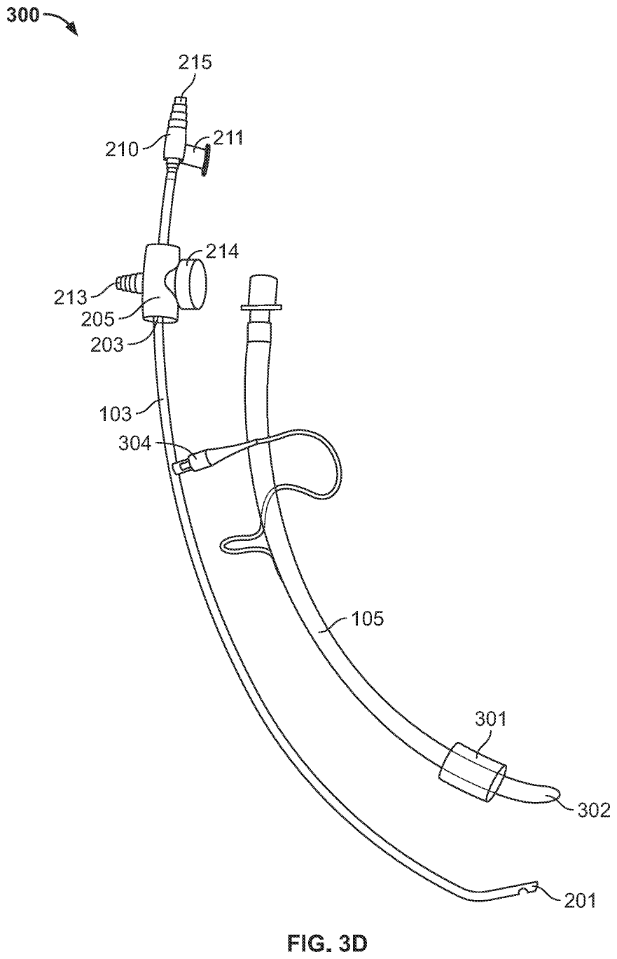

In embodiments, the intubation system 100 comprises a control mechanism located at the proximal end of the catheter 101, and an elongate articulated guide extends from the control mechanism to the distal end of the catheter 101. In embodiments, the endotracheal tube 105 is slidably mounted around the inner tube 103 within the channel 125. In operation, the endotracheal tube 105 slides distally off the tube 103 into the trachea of a patient and is held in place by an inflatable balloon (such as balloon 301 shown in FIG. 3). In various embodiments, the control mechanism is used for controlling the movement of the distal end of the catheter 101. In an embodiment, the control mechanism comprises a handle to control a longitudinal movement of the catheter 101 along the length of the channel 125. During an operation, a medical professional may maneuver the catheter 101 within the patient's body by gripping the handle of the control mechanism or the insertion body 120.

Referring to FIG. 1, in an embodiment, the insertion body 120 and the insertion device/catheter 101 connected to the insertion body 120, are inserted into a patient, distal end first, through the patient's mouth. When properly inserted, the distal end of the outer tube 105 functioning as the endotracheal tube resides in the pharynx of the patient, or more specifically, in the laryngopharynx of the patient. The patient's epiglottis may be supported by the blade 123 in a manner to expose the glottis. The outer tube 105 enables air to be conducted to and from the incapacitated patient. A distal end of the insertion guide body 120 (inside the patient's body) may be moved to see the larynx and the vocal cords of the patient. In an embodiment, the distal end of the insertion guide body 120 is moved to align with the larynx and the vocal cords of the patient. The insertion guide body 120 and tube 105 may be moved distally adjacent the blade 123, so that the tube 105 is also aligned with the larynx and the vocal cords of the patient. When properly aligned, the tube 105 is moved distally along the guide channel 125 of the guide body 120. In various embodiments, the catheter 101 is maneuvered within the channel 125 in order to guide the outer tube 105 past the larynx and the vocal cords of the patient.

In embodiments, the channel 125 within the insertion guide body 120 for supporting the tube assembly of tubes 103, 105 may extend a substantial length of the guide body 120. In an embodiment, the channel 125 has a portion thereof completely enclosed in the guide body 120, with at least a portion thereof being open outwardly of the guide body 120. In embodiments, the guide body 120 defines the channel 125 by extending more than half-way around the guide 125 and tube 103, 105. In an embodiment, the opening in the channel 125 ranges from approximately 10% to 25% of the diameter of the channel 125. In the case of a non-cylindrical channel, the opening is smaller than a side of the guide body 120 and tube 103, 105, such that the guide body 120 and tube 105 are slidably secured in the channel 125. In an embodiment, the channel has a dimension (e.g., diameter) that is more than the dimension (e.g., diameter) of the tube 105 and the guide body 120 to allow the tube 105 to slide within the channel and into the patient. In embodiments, the channel 125 prevents the guide body 120 from articulating within the patient's airway and isolates the movement of the guide body 120 that is outside (e.g., distally past) the channel.

In various embodiments, the intubation system of the present specification is coupled with an imager (not shown in FIG. 1), such as, but not limited to a digital camera (e.g., CCD, CMOS) and/or a light source (e.g., LED) for providing an image of a patient's internal organs surrounding a distal end of the intubation system, when a distal end of the system is inserted into the patient's body. In an embodiment, the imager is located at the distal end of the intubation system and is communicatively coupled with a display screen located at the proximal end of the system via a cable extending through the body of the insertion device of the intubation system, for enabling an operator to view the internal organs (such as the airway) of the patient, when the distal end of the intubation system is inserted into the patient's body.

In various embodiments, the tubes 103, 105, and 206, are made of clear polymer in order to enable visual inspection of the flows therein, during operation of the intubation device.

The intubation system 100 described herein can be used with the intubation device described in U.S. Patent Publication No. 2002/0058599, titled “Endotracheal Intubation Device,” incorporated by reference for any purpose, with the channel and the catheter as described herein. The present intubation system described herein may also include an insufflation system with an air or gas source that is fitted to one of the insertion body 120 or the catheter assembly 101 through an orifice at the proximal end of the intubation system that will remain outside the patient. An example of an insufflation system is described in U.S. Pat. No. 7,458,375, which is assigned to the present assignee and is hereby incorporated by reference for any purpose.

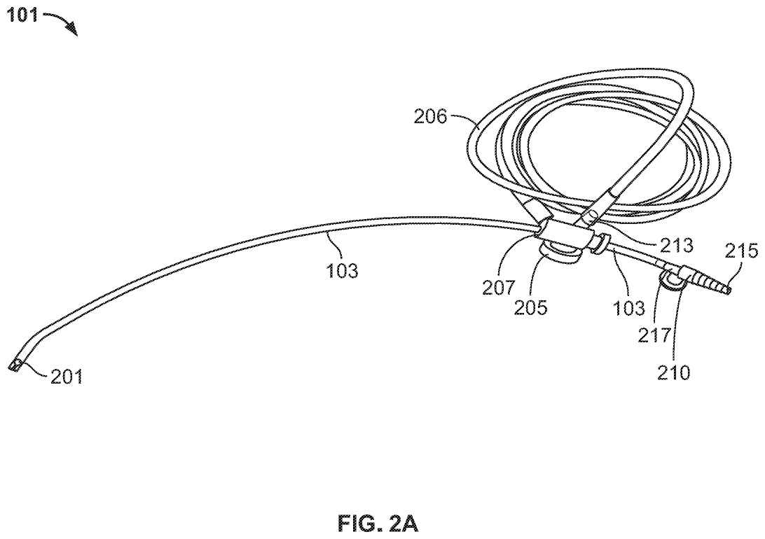

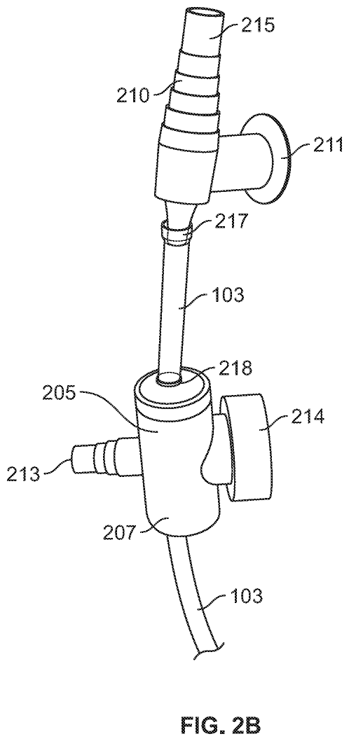

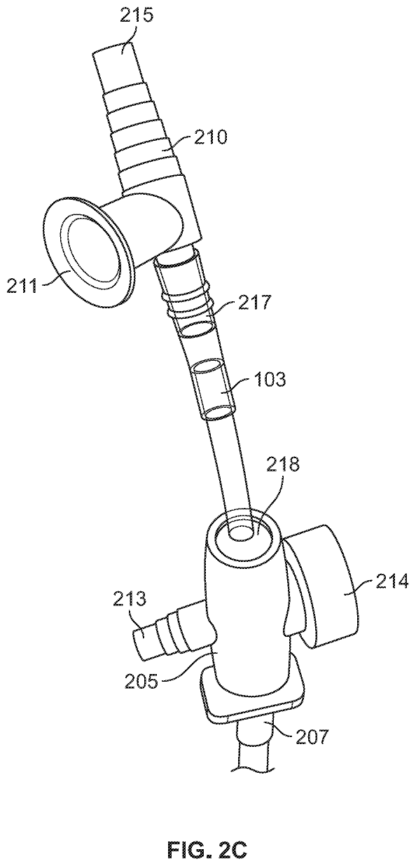

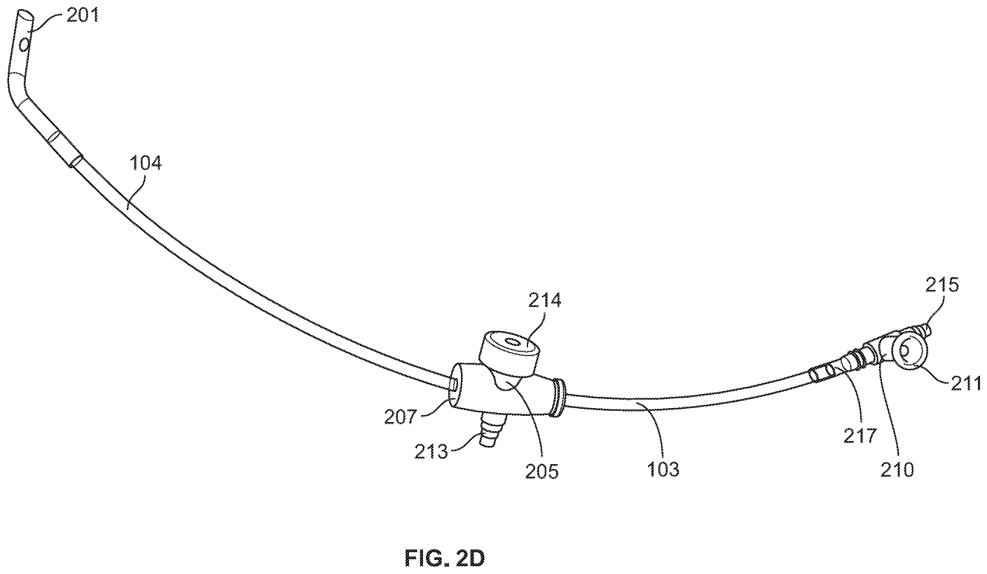

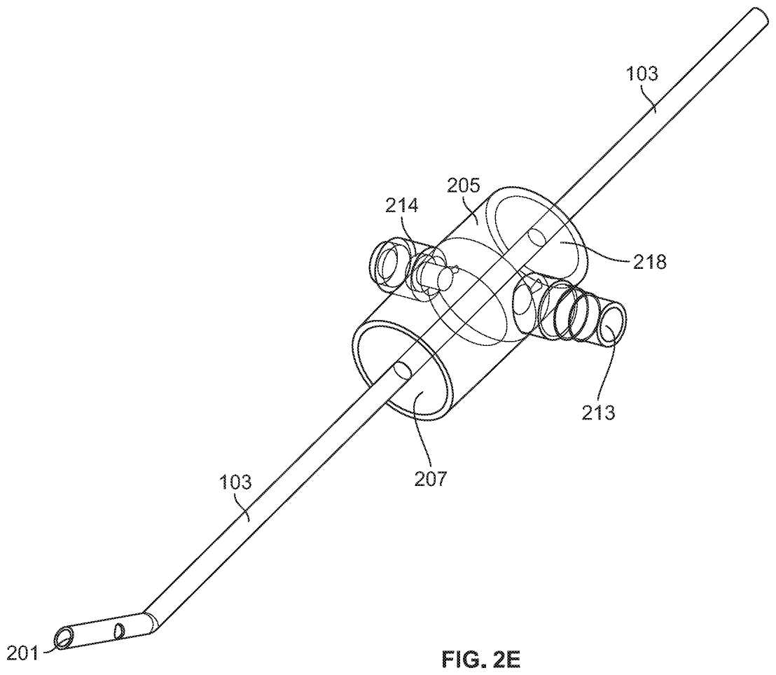

FIG. 2A illustrates a catheter of the intubation system shown in FIG. 1, in accordance with an embodiment of the present specification. FIG. 2B illustrates a close-up view of the connector and hub shown in FIG. 2A. FIG. 2C illustrates another close-up view of the connector and hub shown in FIG. 2B. FIG. 2D illustrates yet another close-up view of the connector and hub shown in FIG. 2B. FIG. 2E illustrates a transparent hub with a steerable catheter, in accordance with an embodiment of the present specification. The catheter 101 shown in FIG. 2A has been illustrated without the endotracheal/outer tube 105 which is shown in FIG. 1 and FIG. 3A-3D.

Referring to FIGS. 1, 2A, 2B, 2C, 2D and 2E, the catheter/inner tube 103 is coupled to a connector 210 and a hub 205 at a proximal end, while a distal end of tube 102 comprises a tip 201. The hub 205 is configured to achieve four critical functions. First, the hub 205 removably attaches to the catheter tube 103 in a leak-proof, yet movable, manner. Second, the hub 205 is configured to allow for an endotracheal tube to be threaded over the catheter 103 and fixedly attach to the hub 205 in a leak-proof manner such that the lumen of the endotracheal tube 105 is in fluid communication with the interior of the hub 205. Third, the hub 205 provides for an enclosed gas connection between an oxygen source and the interstitial space between the outside of the catheter tube 103 and the inside of the endotracheal tube 105. Fourth, the hub has an integrated pressure relief valve that is configured to release pressure above a predesignated pressure level.

More specifically, the hub comprises a proximal end 218 having a circular, sealed opening through which the catheter tube 103 may pass into the hub 205, and a distal end comprising a port 207 for the catheter tube 103 to exit the hub 205 and extend into an endotracheal tube (not shown in FIGS. 2A, 2B, 2C). It should be appreciated that the catheter tube 103 is preferably movable through the hub 205, and therefore not fixedly attached to the proximal end 218 of the hub 205 or the distal port 207. The proximal end 218 of the hub 205 and the distal port 207 seals around the catheter tube 103 by using a friction-based seal. The tube 103 entry and exit openings into and out of the hub 205 are completely air-tight and sealed.

The hub further comprises a connector portion, adjacent to port 207, configured to receive the endotracheal tube 105. The endotracheal tube 105 is threaded over the catheter 103 and connected to the hub 205 such that the interstitial space between the inside of the endotracheal tube 105 and outside of the catheter 103 is in fluid communication with the interior of the hub 205. In one embodiment, the endotracheal tube 105 is connected to the hub 205 using at least one of a friction fit, a threaded fitting, a snap fit, or any other connection mechanism known in the art.

The hub 205 further comprises a port 213, preferably defined by a tapered tube, to connect with a gas supply tube 206. In an embodiment, the gas supply tube 206 is a flexible polymer tube which is transparent, thereby enabling visual inspection of any material within the gas supply tube 206, and remains exterior to the patient. Accordingly, the interior of the hub 205 is in fluid communication with the gas supply tube 206, via port 213, and further in fluid communication with the interstitial space between the inside of the endotracheal tube 105 and outside of the catheter 103.

In embodiments, the hub 205 may further comprise an integrated pressure or flow control valve 214 to limit at least one of a maximum gas pressure that can be applied to the patient or an upper gas flow limit, such as, but not limited to 10 liters per minute. The exit port 207 on the hub 205 allows the tube 103 to exit distally from the hub 205. The hub 205 fluidly separates and seals the tube 103 and the gas supply tube 206 from each other. Each of the ports on the hub 205 are fluidly sealed so that gas or fluids do not escape from the hub 205.

The intubation system may further optionally comprise a connector 210 upstream of the hub 205. The connector 210 comprises a proximal end having a suction control port 215 to control suction pressure at the distal end of the tube 103 and a distal end 217 which is coupled to the proximal end of the tube 103. The suction control port 215 is kept open, as shown in FIGS. 2B, 2C, during operation of the intubation system, in order to prevent a negative pressure at the distal end of the tube 103. When a medical professional operating the intubation device desires suction at the distal end of the tube 103, the suction control port 215 may be closed by using a thumb or finger to cover the port. During operation, the medical professional may have a grip on the port 215 through which they can longitudinally move the tube 103 relative to the hub 205 in order to steer the distal tip 201 of the tube 103 within a patient's body. The connector 210 provides a bayonet 211 or press fit into a port of a suction supply. In an embodiment, the connector 210 can be pressed into a female port in the suction supply.

In an embodiment, the distal end 201 of the tube 103 comprises an angled tip that allows the tube end 201 to be steered within the patient's body when the tube 103 is rotated. In an embodiment, the distal end 201 of the tube 103 is bent at a predefined angle relative to the proximal portion of the main body of the tube 103 to further allow the tube to be steered easily within the patient's body.

FIG. 3A illustrates the intubation device, in accordance with an embodiment of the present specification. FIG. 3B illustrates another view of the intubation device shown in FIG. 3A. FIG. 3C illustrates yet another view of the intubation device shown in FIG. 3B. FIG. 3D illustrates a catheter and an endotracheal tube of the intubation device shown in FIG. 3A separately. Referring to FIGS. 3A, 3B, 3C, and 3D, intubation device 300 comprises a catheter tube 103 coupled with a connector 210 and a hub 205 at a proximal end and comprising an angled distal tip 201. The catheter tube 103 is sheathed by an outer endotracheal tube 105 which comprises a proximal end connected with a port 207 of the hub 205 and a distal end 302. In an embodiment, as shown, the tube 103 extends into the endotracheal tube 105 via the port 207. The hub 205 further comprises a port 213 to connect with a gas supply tube 206. In embodiments, the hub 205 may comprise an integrated pressure relief valve 214 to limit the maximum gas pressure that can be applied to the patient. The connector 210 provides a bayonet 211 or press fit into a port of a suction supply (not shown in the FIGS.). In an embodiment, the connector 210 also comprises a suction control port 215 to control suction pressure at the distal end of the tube 103. In various embodiments, during operation, the distal ends of the tube 103, 105 are inserted with a patient's body.

A balloon 301 is positioned near the distal end 302 of the endotracheal tube 105. The balloon 301 is inflatable through an inflation port 304 when the tube 105 is inserted via a patient's mouth and positioned within the patient's airway. The balloon 301 seals the airway at the patient's trachea and fixes the tube 105 in place within the patient. In an embodiment, once the endotracheal tube 105 is fixed in place, the inner tube 103 may be withdrawn from the patient's body, with the endotracheal tube remaining within the patient.

In various embodiments, the design of the hub 205 enables: the catheter tube 103 to be physically attached in a leak-proof yet movable manner to the hub 205; the endotracheal tube 105 (which is threaded over the catheter 103) to be fixedly attached to the hub 205 in a leak-proof manner; provides for an enclosed gas connection between an oxygen source and the interstitial space between the outside of the catheter tube 103 and the inside of the endotracheal tube 105; and provides for pressure relief above a predefined threshold pressure level, via the pressure relief valve 214.

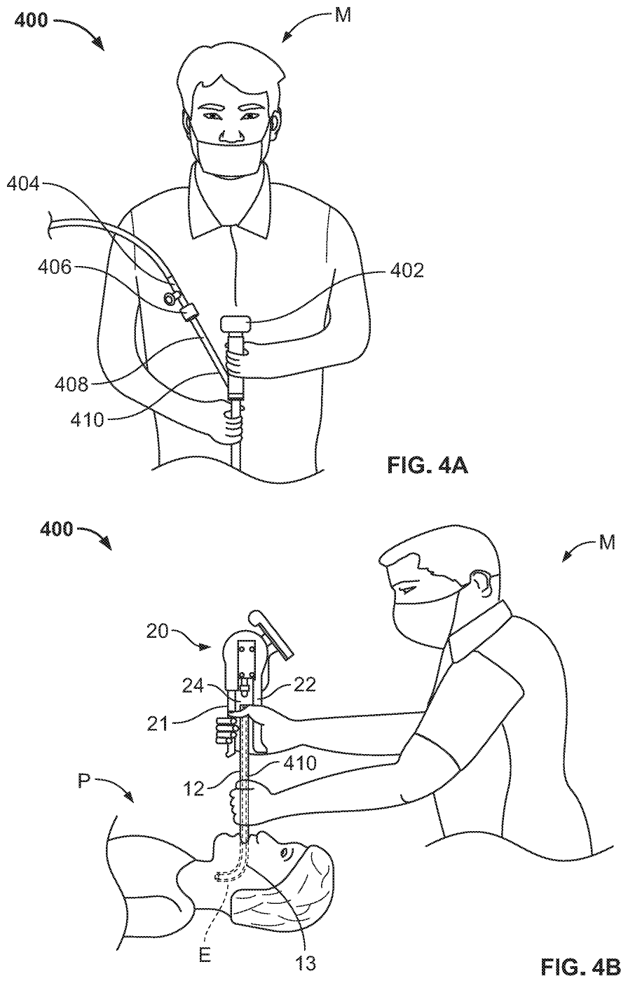

FIG. 4A shows an endotracheal intubation guidance system that may be used with the with the intubation insertion device of the present specification. FIG. 4B shows another view of the endotracheal intubation guidance system shown in FIG. 4A. As can be seen in FIG. 4A, 4B, a medical professional M is operating on a patient P for intubating the patient P via the patient's mouth. In an embodiment, the intubation device 400 of the present specification, may be inserted in the patient's mouth by using an insertion guide system 402. The intubation device 400 comprises a connector 404 and a hub 406 coupled with a proximal end of a catheter 408 sheathed with an endotracheal tube 410. The function and method of operation of the connector 404 and hub 406 have been explained above.

The endotracheal intubation guidance system 402 shown in FIGS. 4A, 4B includes a hand grip 20 with a trigger 21 for convenient articulation of a distal tip 13 of the catheter 408 of the insertion device 400 in use on a patient P, wherein the device 400 is operated by the medical professional M to access the patient's trachea E. In an embodiment, the device 400 is adapted to connect with a hand grip 20 with a proximal end 12 for detachably mounting the device to hand grip 20, and a distal end for entering the trachea E of patient P. The distal tip 13 of the catheter 408 is adapted to curve into trachea E upon actuation of trigger 21 from hand grip 20. The trigger 21 comprising grips 22 and 24 that is squeezed by professional M to actuate the distal tip 13. The endotracheal intubation guidance system 402 is used to insert and place the insertion device 400 into the patient P to clear the trachea E and then catheter 408 is subsequently removed leaving endotracheal tube 410 in place for further procedures to be performed. The suction channel can provide suction at the distal end. The gas channel can provide gas at the distal end.

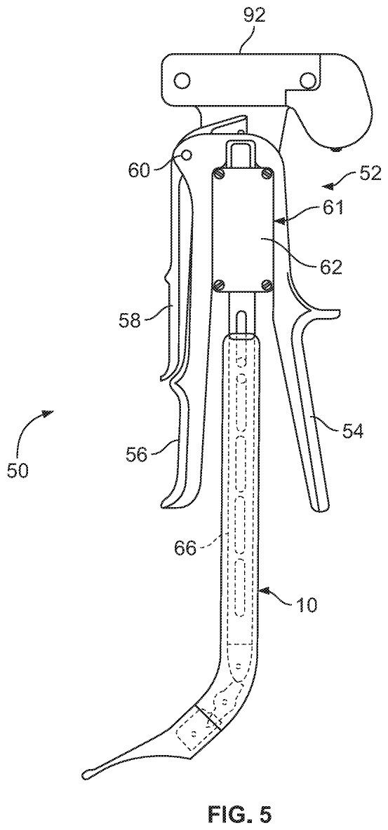

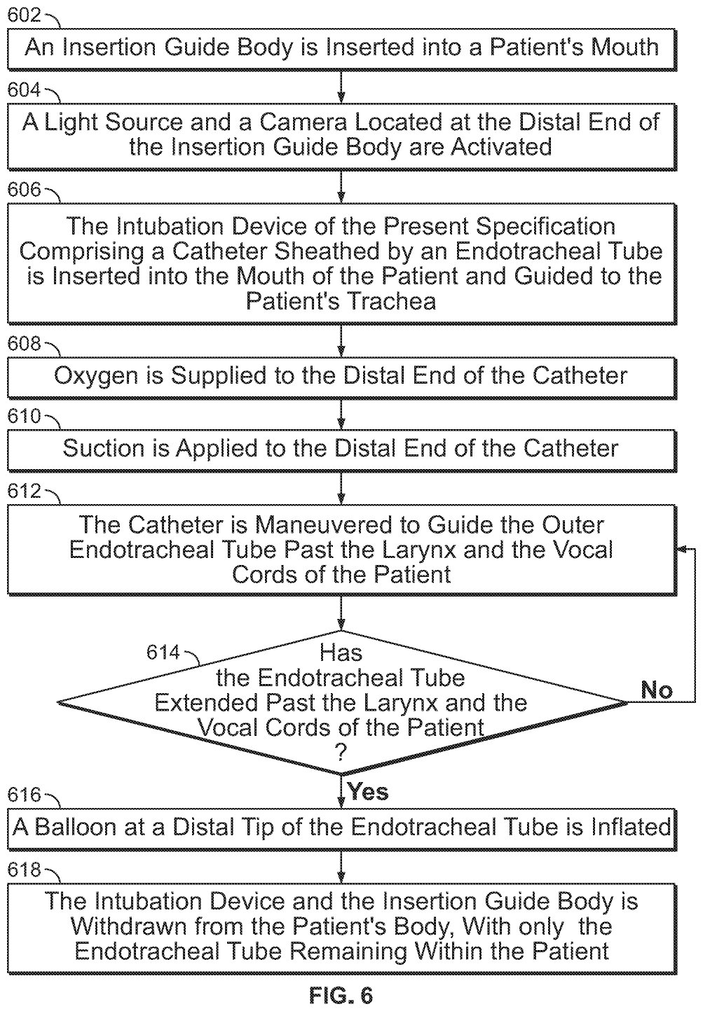

FIG. 5 shows a side view of an endotracheal intubation guidance system that may be used with the with the intubation insertion device of the present specification. The intubation device guidance system 50 has a handle portion 52 including grips 54 and 56, and a lever 58 pivotably connected to handle portion 52 at pivot pin 60. In the illustrated embodiment, referring to FIG. 1 and FIG. 5 an insertion device 61 comprising a stylet assembly 62 functions as the insertion body 120 (shown in FIG. 1). The insertion module 61 houses an actuator assembly operatively connecting lever 58 with a spring loaded member (not shown) reciprocatably movable relative to the longitudinal direction of a jointed arm 66. A sheath 10 is mounted on the insertion body and can define the channel 125 (shown in FIG. 1) for the insertion device 101 (shown in FIG. 1). A display 92 is mounted to the distal end of the intubation device guidance system 50. The present insertion device can be used with the intubation structures and methods described in U.S. patent application Ser. No. 14/501,294, which is hereby incorporated by reference in its entirety. FIG. 6 is a flowchart illustrating the method of operating the intubation device, in accordance with an embodiment of the present specification. At step 602 an insertion guide body is inserted into a patient's mouth. In embodiments, the insertion guide body has a proximal end closer to a medical professional operating upon the patient and a distal end that is designed to be inserted into the patient.

At step 604, a light source and a camera located at the distal end of the insertion guide body are activated. Both of the camera and the light source are electrically connected to a controller located at the proximal end of the insertion guide body. The camera is used to capture images of the internal tissues of the patient, while said tissues are illuminated by the light source. In an embodiment, the camera sends a video signal through electrical connections in the insertion guide body to a viewing device for displaying real time images captured by the camera of the patient's tissues at the distal end of the insertion body, thereby aiding the medical professional in performing the procedure on the patient.

At step 606, the intubation device of the present specification comprising a catheter sheathed by an endotracheal tube is inserted into the mouth of the patient and guided to the patient's trachea by using the insertion guide body and a hub of the intubation device. In an embodiment, the insertion guide body has a generally cylindrical shaped channel in which the catheter is received and guided to the oropharynx and trachea of the patient.

At step 608, oxygen is supplied at the distal end of the catheter by using a gas supply port of the hub. In embodiments, as explained with reference to FIG. 1, the gas supply hub is connected to a gas supply tube, which in turn may be connected to a supply of a gas, such as, but not limited to oxygen. In embodiments, oxygen flowing to distal end of the catheter provides antifogging properties enabling the medical professional performing the procedure to view the internal tissues of the patient's clearly via the camera.

At step 610, suction is applied to the distal end of the catheter by using a suction control port of a connector of the intubation device. As explained above, the catheter is used to suction out any fluids deposited near the distal end of the catheter enabling the medical professional performing the procedure to view the internal tissues of the patient's clearly via the camera.

At step 612, the catheter is maneuvered in order to guide the outer endotracheal tube past the larynx and the vocal cords of the patient. In embodiments, as explained above, the connector of the intubation system and the insertion guide body coupled which may be coupled with an electronic control system are used to guide the distal end of the catheter to reach a desired location with the patient's body. In an embodiment, the insertion guide body and the catheter may be moved distally adjacent the blade, so that the catheter sheathed with the endotracheal tube is aligned with the larynx and the vocal cords of the patient. When properly aligned, the catheter is moved distally along a guide channel of the insertion guide body in order to guide the outer endotracheal tube past the larynx and the vocal cords of the patient.

At step 614, it is determined if the outer endotracheal tube has extended past the larynx and the vocal cords of the patient. At step 616, if the outer endotracheal tube has extended past the larynx and the vocal cords of the patient, a balloon at a distal tip of the endotracheal tube is inflated. In embodiments, the inflated balloon seals the airway at the patient's trachea and fixes the endotracheal tube in place within the patient.

At step 618, the intubation device and the insertion guide body is withdrawn from the patient's body, with only the endotracheal tube remaining within the patient.

The above examples are merely illustrative of the many applications of the system and method of present specification. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

We claim:

1. An intubation device configured to be inserted into a patient's trachea, the intubation device comprising:
a first tube;
a second tube configured to be positioned over the first tube, wherein an interior surface of the second tube and an exterior surface of the first tube define an interstitial space; and
a hub comprising a proximal end, a distal end and a body, wherein the hub comprises:
a gas supply port configured to connect the body of the hub with a gas supply source;
a first port in the proximal end and a second port in the distal end, wherein each of the first port and the second port is configured to receive the first tube and wherein the first tube passes through the body of the hub between the first port and the second port; and
a first connector configured to connect to the second tube such that the interstitial space is in fluid communication with the body and the gas supply port.

2. The intubation device of claim 1, wherein the first port is configured to create a friction-based seal around the exterior surface of the first tube.

3. The intubation device of claim 1, wherein the second port is configured to create a friction-based seal around the exterior surface of the first tube.

4. The intubation device of claim 1, wherein the first tube is a catheter and the second tube is an endotracheal tube.

5. The intubation device of claim 1, wherein the first tube comprises an angled tip configured to be insertable into the patient's trachea via the patient's mouth.

6. The intubation device of claim 1, wherein the hub further comprises a control valve for controlling at least one of a gas flow rate or a level of gas pressure in the body.

7. The intubation device of claim 6, wherein the control valve is configured to limit a gas flow rate to less than 10 liters per minute.

8. The intubation device of claim 6, wherein the control valve is configured to release gas from the body when a pressure of the gas exceeds a predefined threshold value.

9. The intubation device of claim 1, further comprising a suction control port attached to a proximal end of the first tube.

10. The intubation device of claim 9, wherein the suction control port comprises a first connector portion configured to be attached to suction source and an opening configured to direct suction from the suction source through the first tube when the opening is covered and configured to direct suction from the suction source to an external environment when the opening is uncovered.

11. The intubation device of claim 10, wherein the suction control port is configured to be covered by physically blocking said opening.

12. The intubation device of claim 9, wherein, when the suction control port is closed, the suction control port is configured to direct suction through the first tube in order to remove fluids from an oropharynx of the patient.

13. The intubation device of claim 1, wherein, when the gas supply source is configured to supply oxygen.

14. The intubation device of claim 1, wherein the body of the hub is configured to fluidly separate an interior of the first tube from gas supplied by the gas supply source.

15. A method of operating an intubation device configured to be inserted into a patient's trachea, wherein the intubation device comprises a catheter, an endotracheal tube configured to be coaxially positioned over the catheter, wherein an interior surface of the endotracheal tube and an exterior surface of the catheter define an interstitial space, and a hub comprising a proximal end, a distal end and a body, wherein the hub comprises a gas supply port configured to connect the body of the hub with a gas supply source, a first port in the proximal end and a second port in the distal end, wherein each of the first port and the second port is configured to receive the catheter and wherein the catheter passes through the body of the hub between the first port and the second port, and a first connector configured to connect to the endotracheal tube such that the interstitial space is in fluid communication with the body and the gas supply port, the method comprising:
inserting an insertion guide body into the patient's mouth;
inserting the catheter sheathed by the endotracheal tube into the patient's trachea using the insertion guide body;
supplying oxygen to a distal tip of the catheter by passing oxygen from the gas supply source, through the gas supply port, into the body of the hub, and through the interstice;
maneuvering the catheter to guide the endotracheal tube past the larynx and the vocal cords of the patient;
inflating a balloon at a distal tip of the endotracheal tube in order to fix a position of the endotracheal tube at a desired location; and
withdrawing the catheter and the insertion guide body from the patient's body, leaving only the endotracheal tube fixed in the desired location remaining within the patient's body.

16. The method of claim 15, wherein the first port is configured to create a friction-based seal around the exterior surface of the catheter and wherein the second port is configured to create a friction-based seal around the exterior surface of the catheter.

17. The method of claim 15, wherein the distal tip of the catheter comprises an angled tip configured to be insertable into the patient's trachea via the patient's mouth.

18. The method of claim 15, wherein the hub further comprises a control valve for controlling at least one of a flow rate or a level of gas pressure in the body.

19. The method of claim 18, further comprising using the control valve to release gas from the hub when the flow rate of gas exceeds 10 liters per minute.

20. The method of claim 15, further comprising a suction control port attached to a proximal end of the catheter, wherein the suction control port comprises a first connector portion configured to be attached to suction source and a separate opening.

21. The method of claim 20, further comprising modifying an amount of suction being applied from the suction source via the catheter by covering the opening, wherein the suction control port is configured to direct suction from the suction source through the catheter when the opening is covered and configured to direct suction from the suction source to an external environment when the opening is uncovered.

22. The method of claim 15, wherein a distal end of the insertion guide body comprises a light source and a camera.

23. The method of claim 15, wherein the balloon is inflated only after the endotracheal tube has extended past the patient's larynx and vocal cords.

24. The method of claim 15, further comprising steering the distal tip of the catheter by gripping and moving the suction control port attached to the catheter.
